(19) United States
(12) Patent Application Publication
(10) Pub. No.: US 2003/0144255 A1
(43) Pub. Date: Jul. 31, 2003

(54) Compositions for prevention and treatment of dementia

(76) Inventors: Allen I Bain, Vancouver (CA); Alexander Zolotoy, Richmond (CA)

Correspondence Address: Kevin S Lemack, Nields & Lemack, 176 E Main Street, Westboro, MA, 01581, US

(21) Appl. No.: 10/220,836

(22) PCT Filed: Mar. 5, 2001
(86) PCT No.: PCT/CA01/00271

(51) Int. Cl. A61K 31/66; A61K 31/55; A61K 31/522; A61K 31/473; A61K 31/325; A61K 31/137; A61K 31/445; A61K 31/135

(52) U.S. Cl. 514/141; 514/217.01; 514/319; 514/290; 514/297; 514/252.16; 514/646; 514/478; 514/215

(57) ABSTRACT

4,4′-diaminodiphenylsulphone is a bactericide and anti-inflammatory agent. It is known to have therapeutic activity against leprosy, dermatitis herpetiformis, actinomycotic mycetoma, asthma, malaria, rheumatoid arthritis, Kaposiis sarcoma, pneumocystis carinii (pneumonia), subcorneal pustular dermatosis and cystic acne, in patients in need of such therapy. It is also known to have therapeutic activity against memory loss in patients in need of such therapy, including patients suffering from Alzheimer disease and related neurodegenerative disorders. Donepezil hydrochloride (donepezil) is an acetylcholinesterase inhibitor that is currently used for the symptomatic treatment of Alzheimer disease in patients in need of such therapy. It has now been found that combinations of 4,4′-diaminodiphenylsulphone and cholinesterase inhibitors unexpectedly show synergistic effects in the prevention and/or treatment of dementia. The present invention relates to novel compositions and methods of preventing and/or treating dementia using combinations of 4,4′-diaminodiphenylsulphone and a cholinesterase inhibitor (preferably donepezil). The method involves the administration to such individuals a drug composition of 4,4′-diaminodiphenylsulphone and a cholinesterase inhibitor. The invention also relates to a method of preventing and/or treating dementia including senile dementia, that involves the use of this combination of drugs.

### FIELD OF THE INVENTION

The present invention is generally directed toward a pharmaceutical composition and method for the prevention and treatment of dementia which comprises a fixed combination of at least one 4,4′-diaminodiphenylsulphone compound with a cholinesterase inhibitor, although separate compositions of 4,4′-diaminodiphenylsulphone and a cholinesterase inhibitor may be administered together or consecutively or separately to the patient.

### BACKGROUND OF THE INVENTION

4,4′-diaminodiphenylsulphone compounds, especially 4,4′-diaminodiphenylsulfone, are widely used in the pharmaceutical industry. The list of diseases responding to 4,4′-diaminodiphenylsulphone includes dermatitis herpertiformis, leprosy, asthma, malaria, rheumatoid arthritis, pneumonia and pneumocyctis carinii. Recently, it has been reported that 4,4′-diaminodiphenylsulphone is also effective in the prevention and treatment of Alzheimer disease and senile dementia (Lang P. G. J. Am. Acad. Dermatol. 1979, 1, 6: 479-492; McGeer P. L. et al., M. Dementia 1992, 3: 146-149; Coleman M. D. Br. J Dermatology 1993; 129: 507-513.).

In addition to the 4,4′-diaminodiphenylsulphone compounds, a few cholinesterase inhibitors have also been studied for use in the treatment of the symptoms of Alzheimer disease. Two such compounds having cholinesterase inhibitory activity, donepezil and tacrine, are currently prescribed for the symptomatic treatment of patients with mild to moderate symptoms of dementia. These two drugs, however, only offer symptomatic relief of Alzheimer disease and do not stop the progression of the illness; they also have the drawback of hepatotoxicity and/or other cholinergic side effects. The present invention shows that by combining a cholinesterase inhibitor and 4,4′-diaminodiphenylsulphone, an unexpected, synergistic effect is achieved towards the prevention and treatment of dementia.

### SUMMARY OF THE INVENTION

4,4′-diaminodiphenylsulphone is a bactericidal and anti-inflammatory agent that has shown some benefits for preventing and for treating various conditions involving memory loss such as Alzheimer disease and other neurodegenerative disorders (McGeer P.L. et al., Dementia 1992, 3: 146-149). Donepezil is an acetylcholinesterase inhibitor that is currently used for symptomatic treatment of patients with mild to moderate Alzheimer disease. When the two drugs are used in combination, an unexpected, synergistic effect is achieved. Thus, the development of the disease is delayed more than when the individual drug is used separately, and the improvement of the symptoms is more evident than expected from a combination of the two drugs. The present invention relates to a method of preventing and/or treating dementia including senile dementia, using one or more 4,4′-diaminodiphenylsulphone compounds in combination with a compound having cholinesterase inhibitory activity. Also described are pharmaceutical compositions which comprise synergistically effective amounts of at least one 4,4′-diaminodiphenylsulphone compound in combination with a compound having cholinesterase inhibitory activity and methods of using these compositions.

### BRIEF DESCRIPTION OF THE DRAWINGS

Preferred embodiments of the invention will be described in relation to the attached drawing, in which:

FIG. 1 Chemical structure of donepezil hydrochloride, a cholinesterase inhibitor.

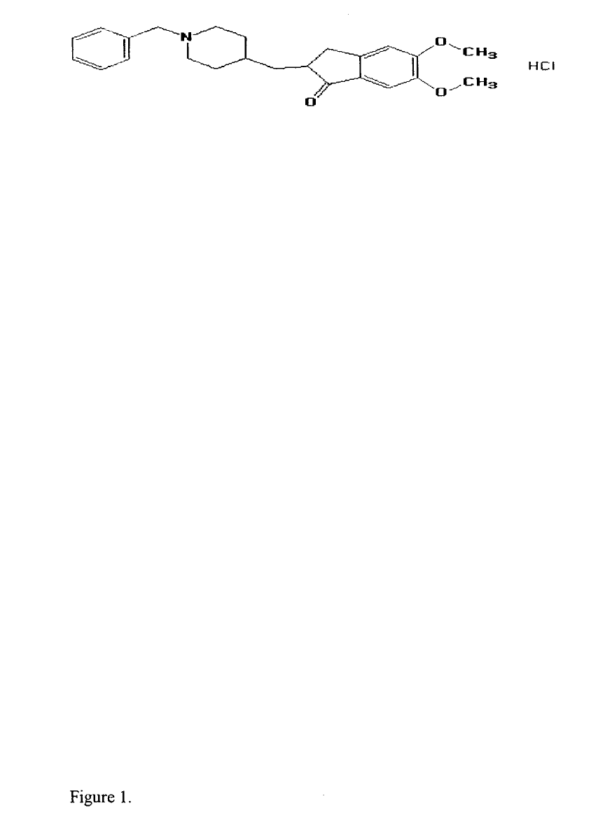

### TERMINOLOGY

The term dementia as used herein includes Alzheimer type dementia, Parkinson type dementia, Huntington type dementia, Pick's type dementia, Creutzfeldt-Jakob type dementia, senile dementia, idiopathic-related dementia, trauma-related dementia, stroke-related dementia, cranial bleed-related dementia, vascular dementia, and includes acute, chronic or recurring forms.

In this patent application, 4,4′-diaminodiphenylsulphone compounds refer to the group of compounds that is closely related to 4,4′-diaminodiphenylsulfone and include but are not limited to 4,4′-diaminodiphenylsulfone, the didextrose sulfonate derivative of 4,4′-diaminodiphenylsulfone (glucosulfone), acedapsone, sulfoxone, sulfetrone, thiazolsulfone, monoacetyldapsone, N-hydroxymonoacetyldapsone, N-hydroxydapsone, and therapeutically and pharmaceutically acceptable salts thereof. Cholinesterase inhibitors such as acetylcholinesterase inhibitors refer to the group of compounds having cholinesterase inhibitory activity and include but are not limited to 3-[1-(phenylmethyl)-4-piperidinyl]-1-(2,3,4,5-tetrahydro-1H-1-benzazepin-8-yl)-1-propanone fumarate and those described in U.S. Pat. No. 5.273,974, the disclosure of which is herein incorporated by reference, 2,3-dihydro-5,6-dimethoxy-2-[[1-(phenylmethyl)-4-piperidinyl]methyl]-1H-inden-1H-one hydrochloride (donepezil) and those described in U.S. Pat. No.4,895,841, the disclosure of which is herein incorporated by reference. (S)-3-[1-(dimethylamino)ethyl]phenyl N-ethyl-N-methylcarbamate (rivastigmine) and those described in U.S. Pat. No. 4,948,807, the disclosure of which is herein incorporated by reference, 9-amino-2,3,5,6,7,8-hexahydro-1H-cyclopenta[b]quinoline (ipidacrine) and those described in U.S. Pat. No. 4,550.113, the disclosure of which is herein incorporated by reference, 1,2,3,4-tetrahydro-9-aminoacridinamine hydrochloride (tacrine), 8-[3-[4-(diethylcarbamoyl)piperazin-1-yl]propyl]-1,3,7-trimethylxanthine hydrochloride (stacofylline) and those described in U.S. Pat. No. 4,599,338, the disclosure of which is herein incorporated by reference, polymorphs of 1-benzyl-4-[5,6-dimethoxy-1-indanon)-2-yl]methylpiperidine hydrochloride as described in U.S. Pat. No. 6,140,321, the disclosure of which is herein incorporated by reference, 4a,5,9,10,11.12-hexahydro-3-methoxy-11-methyl-6-H-benzofuro[3a,3,2-ef][2]benzazepin-6-ol (galanthamine), and dimethyl (2,2,2-trichloro-1-hydroxyethyl)phosphonate (metrifonate), and therapeutically and pharmaceutically acceptable salts thereof. Pharmaceutically and therapeutically acceptable salts include, but are not limited to hydrochloride derivatives, sulphate, phosphate, citrate, fumarate, methanesulphonate, acetate, tartarate, maleate, lactate, mandelate, salicylate, succcinate, methylsulphonic acid derivatives, and cinnamic acid derivatives. Pharmaceutically acceptable excipients include, but are not limited to, sucrose, lactose, glucose, starch, mannitol, sorbitol, cellulose, talc, and cyclodextrins. The binder includes, but is not limited to, cellulose, methylcellulose, polyvinylpyrrolidone, gelatin, gum arabic, polyethylene glycol, sucrose, and starch. The disintegrator includes, but is not limited to, starch, carboxymethylcellulose, and carboxymethylcellulose calcium. The lubricant includes, but is not limited to, talc, etc.

### DETAILED DESCRIPTION OF THE INVENTION

4,4′-diaminodiphenylsulphone compounds have been reported to delay the development and decrease the symptoms of Alzheimer disease, and other forms of dementia including senile dementia (McGeer P.L. et al., Dementia 1992, 3: 146-149). A number of cholinesterase inhibitors has also been studied for use in the treatment of the symptoms of Alzheimer disease. donepezil, an acetylcholinesterase inhibitor, is known to be effective in treating the symptoms of Alzheimer disease. The inventors have unexpectedly discovered that when administered in combination, 4,4′-diaminodiphenylsulphone and a cholinesterase inhibitor, preferably donepezil, have a synergistic effect on preventing and/or treating the symptoms of dementia in patients in need of such therapy. The present invention is directed to novel pharmaceutical compositions for the prevention and/or treatment dementia. In particular, it relates to novel therapeutic compositions that comprises one or more 4,4′-diaminodiphenylsulphone compounds in combination with a compound having cholinesterase inhibitory activity for use in the prevention and/or treatment of dementia. In one embodiment of the present invention, the 4,4′-diaminodiphenylsulphone compound is selected from the group consisting of 4,4′-diaminodiphenylsulfone, the didextrose sulfonate derivative of 4,4′-diaminodiphenyisulfone (glucosulfone), acedapsone, sulfoxone, sulfetrone, thiazolsulfone, monoacetyldapsone, N-hydroxymonoacetyldapsone, N-hydroxydapsone, and therapeutically and pharmaceutically acceptable salts thereof. In another embodiment of the present invention, the compound having cholinesterase inhibitory activity is selected from the group consisting of 3-[1-(phenylmethyl)-4-piperidinyl]-1-(2,3,4,5-tetrahydro-1H-1-benzazepin-8-yl)-1-propanone fumarate, 2,3-dihydro -5,6-dimethoxy-2-[[1-(phenylmethyl)-4-piperidinyl]methyl]-1H-inden-1-one hydrochloride (donepezil), (S)-3-[1-(dimethylamino)ethyl]phenyl N-ethyl-N-methylcarbamate (rivastigmine), 9-amino-2,3,5,6,7,8-hexahydro-1H-cyclopenta[b]quinoline (ipidacrine), 1,2,3,4-tetrahydro-9-aminoacridinamine hydrochloride (tacrine) in combination with dimethyl (2,2,2-trichloro-1-hydroxyethyl)phosphonate (metrifonate), 8-[3-[4-(diethylcarbamoyl)piperazin -1-yl]propyl]-1,3,7-trimethylxanthine hydrochloride (stacofylline), 4a,5,9,10,11,12-hexahydro-3-methoxy-11-methyl-6-H-benzofuro[3a,3,2-ef][2]benzazepin -6(galanthamine) and therapeutically and pharmaceutically acceptable salts thereof.

In one preferred embodiment, the 4,4′-diaminodiphenylsulphone compound in the pharmaceutical compositions of the present invention is 4,4′-diaminodiphenylsulfone. In another preferred embodiment, the compound having cholinesterase inhibitory activity in the pharmaceutical compositions of the present invention is 2,3-dihydro-5,6-dimethoxy-2-[[1-(phenylmethyl) -4-piperidinyl]methyl]-1H-inden-1-one hydrochloride (donepezil). In yet another preferred embodiment, the pharmaceutical composition of the present invention is a combination of 4,4′-diaminodiphenylsulfone with a cholinesterase inhibitor (preferably donepezil).

Another aspect of the present invention provides the use of the above-described pharmaceutical compositions in a manufacture of a medicament.

Another aspect of the present invention provides the use of the above-described pharmaceutical compositions in a method for treating or preventing dementia in a mammal in need thereof, which comprises administering to such mammal a therapeutically effective amount of one of the above-described pharmaceutical compositions.

Another aspect of the present invention provides a method for treating or preventing dementia in a mammal in need thereof, which comprises administering to such mammal synergistically effective amounts of at least one 4,4′-diaminodiphenylsulphone compound in combination with a compound having cholinesterase inhibitory activity.

The invention further provides a method for treating or preventing dementia in a mammal in need thereof, which comprises administering to such mammal synergistically effective amounts of at least one 4,4′-diaminodiphenylsulphone compound in combination with a compound having cholinesterase inhibitory activity, wherein the 4,4′-diaminodiphenylsulphone compound is selected from the group consisting of 4,4′-diaminodiphenylsulfone, the didextrose sulfonate derivative of 4,4′-diaminodiphenylsulfone (glucosulfone), acedapsone, sulfoxone, sulfetrone, thiazolsulfone, monoacetyldapsone, N-hydroxymonoacetyldapsone, N-hydroxydapsone, and therapeutically and pharmaceutically acceptable salts thereof.

The invention also provides a method for treating or preventing dementia in a mammal in need thereof, which comprises administering to such mammal synergistically effective amounts of at least one 4,4′-diaminodiphenylsulphone compound in combination with a compound having cholinesterase inhibitory activity, wherein the compound having cholinesterase inhibitory activity is selected from the group consisting of 3-[1-(phenylmethyl)-4-piperidinyl]-1-(2,3,4,5-tetrahydro-1H-1-benzazepin-8-yl)-1-propanone fumarate, 2,3-dihydro-5,6-dimethoxy-2-[[1-(phenylmethyl)-4-piperidinyl]methyl]-1H-inden-1-one hydrochloride (donepezil), (S)-3-[1-(dimethylamino)ethyl ]phenyl N-ethyl-N-methylcarbamate (rivastigmine), 9-amino-2,3,5,6,7,8-hexahydro-1H-cyclopenta[b]quinoline (ipidacrine), 1,2,3,4-tetrahydro-9-aminoacridinamine hydrochloride (tacrine), 8-[3-[4-(diethylcarbamoyl)piperazin-1-yl]propyl]-1,3,7-trimethylxanthine hydrochloride (stacofylline), 4a,5,9,10,11,12-hexahydro-3-methoxy-11-methyl-6-H-benzofuro[3a,3,2-ef][2]benzazepin-6-ol (galanthamine), and dimethyl (2,2,2-trichloro-1-hydroxyethyl)phosphonate (metrifonate), and therapeutically and pharmaceutically acceptable salts thereof.

In a preferred embodiment, in the above described methods of the present invention of the present invention, the compounds having cholinesterase inhibitory activity are combinations of 1,2,3,4-tetrahydro-9-aminoacridinamine hydrochloride (tacrine) in combination with dimethyl-(2,2,2-trichloro-1-hydroxyethyl)phosphonate (metrifonate).

In another preferred embodiment, in the above-described methods of the present invention, the 4,4′-diaminodiphenylsulphone compound is 4,4′-diaminodiphenylsulfone.

In another preferred embodiment, in the above-described methods of the present invention, the compound having cholinesterase inhibitory activity is 2,3-dihydro-5,6-dimethoxy-2-[[1-(phenylmethyl) -4-piperidinyl]methyl]-1H-inden-1-one hydrochloride (donepezil).

In yet another preferred embodiment, in the above-described methods of the present invention, a combination of 4,4′-diaminodiphenylsulfone with 2,3-dihydro-5,6-dimethoxy-2-[[1-(phenylmethyl) -4-piperidinyl]methyl]-1H-inden-1-one hydrochloride (donepezil) is administered.

In yet a further preferred embodiment, in the above described methods of the present invention, a combination of 4,4-diaminodiphenylsulphone compound(s) in combination with cholinesterase inhibitor(s) in effective dosage unit forms.

The pharmaceutical compositions of the present invention can be formulated for, oral administration, inhalation devices, depot, intra-adipose, intravenously, sublingually, perilingually, subcutaneously, rectally, or transdermally, or by any other medically-acceptable means, but preferably orally by mixing each of the above compounds with a pharmacologically acceptable carrier or excipient. Orally administered drugs of the present invention overcome several obstacles to reach their desired targets as compared to rectal administration in the form of modified-release suppositories. The amount of active ingredient(s) that may be combined with desired carrier material(s) to produce single or multiple dosage forms will vary depending upon the host in need thereof and the respective mode of administration. For example, a formulation intended for oral administration of humans may contain from 0.0 mg to 500 mg of active agent(s) compounded with an appropriate convenient amount of carrier material which may vary in composition from about 1 to 99 percent of total composition. Before orally administered drugs enter the general circulation of the human body, they are absorbed into the capillaries of the upper gastrointestinal tract and are transported by the portal vein to the liver. The enzymatic activities, the pH found in gastrointestinal fluids or tissues, the concurrent intake of food and consequent agitation may inactivate the drug or cause the drug to dissolve poorly and consequently, decrease compliance, increase the risk of side effects and substantially reduce the efficacy of the drug. Varying dosage unit forms of the present invention comprise at least one 4,4′-diaminodiphenylsulphone compound in combination with a compound having cholinesterase inhibitory activity as active ingredients and have surprisingly shown an increase in the efficacy and for inhibiting the progression of dementia and/or for treating the disease. This is an unexpected finding in that in many cases the decreased bioavailability of orally administered drugs is a consequence of this “first pass” effect. In addition, following absorption in the intestine, orally administered drugs that are subjected to a “first pass” clearance by the liver e.g., cholinesterase inhibitors maybe excreted into bile or converted into pharmacologically inactive or active metabolites thereby decrease compliance, increase the risk of side effects and substantially reduce the efficacy of the drug(s) for the drug(s) intended targets.

The pharmaceutical composition(s) of the present invention for inhibiting the progression of dementia and/or for treating the disease, comprise at least one 4,4′-diaminodiphenylsulphone compound in combination with a compound having cholinesterase inhibitory activity as active ingredients in dosage unit form(s). In cases where the biological half-life of the cholinesterase inhibitor is different than that of the 4,4′-diaminodiphenylsulphone compounds, it may be advantageous to administer the drugs in separate or admixed compositions and a controlled release composition may be used for the active compound(s) with the shortest biological half-life. Alternatively, a tablet composition may be used that allows for fast release of the compound(s) with the longest duration and delayed release of the compound(s) with the shortest duration of activity.

The dosage unit forms will generally contain between from about 0.0 mg, 0.5., 1.0, 3.0, 5.0 mg or 10 mg of cholinesterase inhibitor and from about 15, 30 mg, 40 mg, 45 mg, 55 mg, 60 mg, 80 mg, 100 mg, 130 mg, 170 mg, 250 mg, 330 mg, 450 mg or 500 mg of 4,4′-diamiondiphenylsulphone and mixtures thereof.

The pharmaceutical composition for treating or preventing dementia of the present invention can be provided, for example, in the alternative forms prepared by the following procedures: (1) the above compounds are mixed optionally with a pharmaceutically acceptable excipient or the like by procedures known in the art to provide one dosage form, (2) the respective compounds are independently processed, optionally together with a pharmaceutically acceptable excipient or the like, to use in combination with independent dosage forms, or (3) the respective compounds are independently processed, optionally together with a pharmaceutically acceptable excipient or the like, to provide independently prepared dosage forms as a set. The preferred dosage unit forms will generally contain between from about 0.01, 0.5, 1.0, 5.0, 15, 30 mg, 40 mg, 50 mg or 100 mg.

If the respective compounds are independently processed to provide independently prepared dosage forms, each compound of the pharmaceutical composition of the present invention may be administered to one patient or a prospective patient concurrently or consecutively, and the quantity and period of dosing of the respective compounds need not be the same.

The pharmaceutical composition of the present invention for treating and/or preventing dementia, can be provided in any and all dosage forms that can be administered to patients by the oral route, such as tablets, fine granules, capsules, and granules, and others. Preferred forms are tablets.

The pharmaceutical composition of the present invention may be manufactured using an excipient, binder, disintegrator, lubricant, and/or other formulation additives. The composition may be provided in sustained release dosage forms. The dosage forms may be manufactured by coating the tablets, granules, fine granules, capsules, etc. with oleaginous substances including, but not limited to triglycerides, polyglycerol fatty acid esters and hydroxypropylcellulose.

The pharmaceutical composition containing 4,4′-diaminodiphenylsulphone, for instance, can be provided in various dosage forms in accordance with procedures known in the art such as those described in Yuasa, Y. Yakugaku Zasshi 1997; 117(10-11): 957-62, or any pharmaceutical procedures analogous thereto. Among others, dosage forms containing 5 to 100 mg of 4,4′-diaminodiphenylsulphone, preferred are tablets containing 25 mg of 4,4′diaminodiphenylsulphone and dosage forms containing from about 0.01 to 10 mg of a cholinesterase inhibitor. Preferred are capsules containing from about 4.5 to 10 mg of donepezil.

The pharmaceutical composition of the present invention for preventing and/or treating dementia are useful for treating and/or preventing and/or inhibiting the progression of all forms of dementia as described herein.

The suggested dosage of 4,4′-diaminodiphenylsulphone is about 1 mg/kg/day. The dosage may be adjusted according to the symptomatic severity of dementia and other medical conditions of the patient.

The suggested dosage of the compound having cholinesterase inhibitory activity is dependent on the particular species of compound used, but may be below the threshold of peripheral nervous symptoms, such as parasympathetic effects (e.g. diarrhea, tearing, watery mouth, etc.). When 3-[1-(phenylmethyl)-4-piperidinyl]-1-(2,3,4,5-tetrahydro-1H-1-benzazepin-8-yl)-1-propanone fumarate is employed, its dosage is about 1 mg to about 4 mg/kg/day, preferably about 0.1 mg/kg/day to about 2 mg/kg/day. When 2,3-dihydro-5,6-dimethoxy-2-[[1 -(phenylmethyl) -4-piperidinyl]methyl]-1H-inden-1-one hydrochloride (donepezil) is employed, its suggested dosage is 1 mg to 200 mg per day, the preferred dosage is 0.07 mg/kg/day to 0.7 mg/kg/day. When tacrine is employed, its suggested dosage is about 0.13 mg/kg/day to about 6.7 mg/kg/day, preferably about 0.7 mg/kg/day to about 3 mg/kg/day. When ipidacrine is employed, its suggested dosage is about 0.13 mg/kg/day to about 6.7 mg/kg/day, preferably about 100 mg to about 4 mg/kg/day. When (S)-3-[1-(dimethylamino)ethyl]phenyl N-ethyl-N-methylcarbamate (rivastigmine) is employed, its suggested dosage is about 0.01 mg/kg/day to about 0.7 mg/kg/day, preferably about 0.07 mg/kg/day to about 0.25 mg/kg/day. When 8-[3-[4-(diethylcarbamoyl)piperazin-1-yl]propyl]-1,3,7-trimethylxanthine hydrochloride (stacofylline) is employed, its suggested dosage is about 0.01 mg/kg/day to about 7 mg/kg/day, preferably about 0.13 mg/kg/day to about 2.5 mg/kg/day. The dosage may be adjusted according to the symptomatic severity of dementia and other medical conditions of the patient.

The pharmaceutical composition of the present invention for treating and/or preventing dementia, may be used in combination with various compatible medicaments such as centrally acting drugs e.g. antianxiety drugs, sleep inducing agents, therapeutic agents for schizophrenia, antiparkinsonian drugs, nootropic agents (e.g. brain circulation improving agents, cerebral metabolism activators, etc.). antihypertensive agents, antidiabetics, antihyperlipidemic drugs, nutritional supplements (e.g. vitamins. etc.), digestants and absorption promoters, gastrointestinal drugs, in addition to the 4,4′-diaminodiphenylsulphone compounds and the compound having cholinesterase inhibitory activity.

The following test and formulation examples are further illustrative of the present invention.

### EXAMPLES

### Experimental Example

The ameliorative effect of the combined use of the 4,4′-diaminodiphenylsulphone compounds with the compounds having cholinesterase inhibitory activity on learning deficits was investigated in aged rats. The following methods describe a set of experiments using the combination of 4,4-diaminodiphenylsulphone with a cholinesterase inhibitor.

Methods

Male (3 to 27 months old) rats of transgenic strain were used. The aged rats were divided into the following four groups.

1) Control group: Repeated administration of placebo pill.

2) 4,4′-diaminodiphenylsulphone group: Repeated oral administration of 4,4 -diaminodiphenylsulphone 1 mg/kg.

3) Cholinesterase inhibitor group: Repeated oral administration of donepezil 0.3 mg/kg.

4) Combination group: Repeated oral administration of 4,4′-diaminodiphenylsulphone 3 mg/kg and donepezil 0.3 mg/kg.

In the combination group, 4,4′-diaminodiphenylsulphone was administered 30 minutes after administration of donepezil.

Passive avoidance learning test was started on day 14 of treatment, and Morris water maze learning test on day 20 of treatment.

On each day of experiment, 4,4′-diaminodiphenylsulphone and a cholinesterase inhibitor were administered 30 minutes and 1 hour, respectively, before initiation of the trial.

1. Passive Avoidance Learning

The passive avoidance learning test was performed using a chamber consisting of light and dark compartments. Young rats (pill, 10 animals) and aged rats (control group, 10 animals; 4,4′-diaminodiphenylsulphone group, 10 animals; donepezil group, 10 animals; combination group, 10 animals) were individually placed in the light compartment and 10 seconds later, the sliding door was opened. After a mouse moves to the dark compartment, the mouse was kept there for about 10 seconds with the door closed. One to two hours after the habituation trial, acquisition trial was performed.

In the acquisition trial, after a mouse moved to the dark compartment, a foot shock (0.4 mA, 3 seconds) was given through the grid floor. Retention trials are performed 24 hours after acquisition trials.

In each trial, the latency from opening of the slide door until the animal moved to the dark compartment (step-through latency) was measured.

2. Morris Water Maze Learning

Same animals used in the passive avoidance test were subjected for the water maze task. However, some rats can not swim well in the water tank, thus they were excluded in the water maze task. The water maze learning test was performed on young rats (saline, 10 animals) and aged rats (control group, 9 animals; 4,4′-diaminodiphenylsulphone group, 9 animals; Compound B group, 8 animals; combination group, 8 animals).

In pretraining which was performed for swimming training and motivation for escaping from water, four trials were performed using a water bath, 80 cm in diameter, in a condition that the platform was visible. From the following day, using a water bath, 120 cm in diameter, learning trials, one session (four trials) per day, were performed with the platform being placed below the water.

Results

1. Passive Avoidance Learning

The control group showed a significant decrease in the avoidance time as compared with the young group. The 4,4′-diaminodiphenylsulphone group or the donepezil group showed significant improvement of the learning deficit in aged rats. The combination group, however, showed a much higher and significant improvement compared with 4,4′-diaminodiphenylsulphone or donepezil groups.

These results indicate that the combination of 4,4′-diaminodiphenylsulphone and donepezil improves the learning deficit in aged rats, and has a greater effect that the use of either drug alone.

2. Water Maze Learning

In the water maze task, the control group showed a significant prolongation of latency to find platform submerged in the water compared with the young rats. The 4,4′-diaminodiphenylsulphone group and the donepezil group showed a significant improvement in water maze learning deficit. However, the combination group showed a significant shortening of latency compared with the control, 4,4′-diaminodiphenylsulphone and donepezil groups.

These results indicate that combination of 4,4′-diaminodiphenylsulphone and donepezil improve water maze learning deficit in aged rats, and this effect is greater than that seen when either drug is used alone.

Other tests may be performed using animal models of dementia such as some of those described and reviewed in the following references: Higgins L. S., Mol. Med Today 1999, 5(6):274-6; Borchelt D. R. et al., Brain Pathol. 1998, 8(4):735-57 and Guenette S. Y. et al., Neurobiol. Aging 1999, 20(2):201-11.

### Preparation Example 1

Production of 1000 tablets, each containing 25 mg of 4,4′-diaminodiphenylsulphone

| | |
|---|---|
| 4,4′-diaminodiphenylsulphone | 25.000 g |
| Lactose (EP) | 233.186 g |
| Gelatinized starch | 11.210 g |
| Calcium salt of carboxymethyl- | 67.270 g |
| cellulose (ECG 505) | |
| Magnesium stearate (EP) | 1.120 g |
| Hydroxypropylmethyl cellulose | |
| USP (Pharmacoat 606) | 5.573 g |
| Rolyethylene glycol (NF 6000) | 1.393 g |
| Propylene glycol (EP) | 0.465 g |
| Talc (EP) | 1.858 g |
| Titanium oxide (EP E171) | 2.786 g |
| Red Color 30 (E172) | 0.139 g |
| Total | 350.000 g |

After 4,4′-diaminodiphenylsulphone and water were added to, and kneaded with, the above excipients for pharmaceutical preparations, the mixture was dried. To this dry kneaded product, the above disintegrants and lubricant were added, followed by uniform mixing, after which the whole mixture is compressed using a compressive tableting machine to yield 1,000 tablets 11 mm in diameter, 4.3 mm in thickness and 350 mg in weight which contained 25 mg of 4,4′-diaminodiphenylsulphone per tablet.

### Preparation Example 2

Production of 1000 tablets, each containing 0.6 mg donepezil

| | |
|---|---|
| Donepezil | 0.60 g |
| Lactose | 19.00 g |
| Cornstarch | 50.00 g |
| Magnesium stearate | 2.000 g |
| Total | 72.00 g |

The above donepezil, lactose, and corn starch (20 g) were blended. This blend was granulated with a paste prepared from corn starch (15 g) and water (25 ml). After addition of corn starch (15 g) and magnesium stearate (2 g), the granulation was compressed with a tablet machine to provide 2000 tablets (3 mm in diameter) each containing 0.3 mg of donepezil.

### INDUSTRIAL APPLICABILITY

The mixture, combination dosage form, or concomitant therapy which comprises one or more 4,4′-diaminodiphenylsulphone compounds in combination with a compound having cholinesterase inhibitory activity can be safely administered or applied to patients with dementia, and may be used to prevent and/or treat the symptoms of these diseases.

As will be apparent to those skilled in the art in the light of the foregoing disclosure, many alterations and modifications are possible in the practice of this invention without departing from the spirit or scope thereof.

What is claimed is:

1. A pharmaceutical composition which comprises at least one 4,4′-diaminodiphenylsulphone compound or salt thereof in combination with a compound having cholinesterase inhibitory activity or salt thereof and a pharmaceutically acceptable carrier, diluent or excipient.

2. A composition of claim 1, wherein the 4,4′-diaminodiphenylsulphone compound is selected from the group consisting of 4,4′-diaminodiphenylsulfone, the didextrose sulfonate derivative of 4,4′-diaminodiphenylsulfone (glucosulfone), acedapsone, sulfoxone, sulfetrone, thiazolsulfone, monoacetyldapsone, N-hydroxymonoacetyldapsone, N-hydroxydapsone, and therapeutically and pharmaceutically acceptable salts thereof.

3. A composition of claim 1, wherein the 4,4′-diaminodiphenylsulphone compound is 4,4′-diaminodiphenylsulfone.

4. A composition of claims 1, 2 or 3, wherein the compound having cholinesterase inhibitory activity is selected from the group consisting of 3-[1-(phenylmethyl)-4-piperidinyl]-1-(2,3,4,5-tetrahydro -1H-1-benzazepin-8-yl)-1-propanone fumarate, 2,3-dihydro-5,6-dimethoxy-2-[[1-(phenylmethyl) -4-piperidinyl]methyl]-1H-inden-1-one hydrochloride (donepezil), (S)-3-[1-(dimethylamino)ethyl]phenyl N-ethyl-N-methylcarbamate (rivastigmine), 9-amino-2,3,5,6,7,8-hexahydro-1H-cyclopenta[b]quinoline (ipidacrine), 1,2,3,4-tetrahydro-9-aminoacridinamine hydrochloride (tacrine), 8-[3-[4-(diethylcarbamoyl)piperazin-1-yl]propyl]-1,3,7-trimethylxanthine hydrochloride (stacofylline), 4a,5,9,10,11,12-hexahydro-3-methoxy-11-methyl-6-H-benzofuro[3a,3,2-ef][2]benzazepin-6-ol (galanthamine), and dimethyl (2.2.2-trichloro -1-hydroxyethyl)phosphonate (metrifonate), and therapeutically and pharmaceutically acceptable salts thereof.

5. A composition of claim 4, wherein the compound having cholinesterase inhibitory activity is 3-[1-(phenylmethyl)-4-piperidinyl]-1-(2,3,4,5-tetrahydro-1H-1-benzazepin-8-yl)-1-propanone fumarate.

6. A composition of claim 4, wherein the compound having cholinesterase inhibitory activity is 2,3-dihydro-5,6-dimethoxy-2-[[1-(phenylmethyl)-4-piperidinyl]methyl]-1H-inden-1-one hydrochloride (donepezil).

7. A composition of claim 4, wherein the compound having cholinesterase inhibitory activity is (S)-3-[1-(dimethylamino)ethyl]phenyl N-ethyl-N-methylcarbamate (rivastigmine).

8. A composition of claim 4, wherein the compound having cholinesterase inhibitory activity is 1,2,3,4-tetrahydro-9-aminoacridinamine hydrochloride (tacrine).

9. A composition of claim 4, wherein the compound having cholinesterase inhibitory activity is dimethyl-(2,2,2-trichloro-1-hydroxyethyl)phosphonate (metrifonate).

10. A method for treating or preventing dementia in a mammal in need thereof, which comprises administering to such mammal an effective amount of at least one 4,4′-diaminodiphenylsulphone compound or salt thereof in combination with a compound having cholinesterase inhibitory activity or salt thereof.

11. The method according to claim 10, wherein the 4,4′-diaminodiphenylsulphone compound is selected from the group consisting of 4,4′-diaminodiphenylsulfone, the didextrose sulfonate derivative of 4,4′-diaminodiphenylsuifone (glucosulfone), acedapsone, sulfoxone, sulfetrone, thiazolsulfone, monoacetyldapsone, N-hydroxymonoacetyldapsone, N-hydroxydapsone, and therapeutically and pharmaceutically acceptable salts thereof.

12. The method according to claim 10, wherein the 4,4′-diaminodiphenylsulphone compound is 4,4′-diaminodiphenylsulfone in the dosage range of 5 mg to 400 mg once or twice a day.

13. The method according to claim 10, 11 or 12, wherein the compound(s) having cholinesterase inhibitory activity is selected from the group consisting of 3-[1-(phenylmethyl)-4-piperidinyl]-1-(2,3,4,5-tetrahydro-1H-1-benzazepin-8-yl)-1-propanone fumarate, 2,3-dihydro -5,6-dimethoxy-2-[[1-(phenylmethyl)-4-piperidinyl]methyl]-1H-inden-1-one hydrochloride (donepezil), (S)-3-[1-(dimethylamino)ethyl]phenyl N-ethyl-N-methylcarbamate (rivastigmine), 9-amino-2,3,5,6,7,8-hexahydro-1H-cyclopenta[b]quinoline (ipidacrine), 1,2,3,4-tetrahydro-9-aminoacridinamine hydrochloride (tacrine), 8-[3-[4-(diethylcarbamoyl)piperazin-1-yl]propyl]-1,3,7-trimethylxanthine hydrochloride (stacofylline), 4a,5,9,10,11,12-hexahydro-3-methoxy-11-methyl-6-H-benzofuro[3a,3,2-ef][2]benzazepin-6-ol (galanthamine), and dimethyl (2,2,2-trichloro-1-hydroxyethyl)phosphonate (metrifonate), and therapeutically and pharmaceutically acceptable salts thereof.

14. The method according to claim 13 wherein the preferred dosage range for the cholinesterase inhibitory activity will be 0.01 to 4.5 mg once or twice a day.

15. The method according to claim 13, wherein the compound having cholinesterase inhibitory activity is 3-[1-(phenylmethyl)-4-piperindinyl]-1-(2,3,4,5-tetrahydro-1H-1-benzazepin-8-yl)-1-propanone fumarate.

16. The method according to claim 13, wherein the compound having cholinesterase inhibitory activity is 2,3-dihydro-5,6-dimethoxy-2-[[1-(phenylmethyl)-4-piperidinyl]methyl]-1H-inden-1-one hydrochloride (donepezil).

17. The method according to claim 13, wherein the compound having cholinesterase inhibitory activity is (S)-3-[1-(dimethylamino)ethyl]phenyl N-ethyl-N-methylcarbamate (rivastigmine).

18. The method according to claim 13, wherein the compound having cholinesterase inhibitory activity is 1,2,3,4-tetrahydro-9-aminoacridinamine hydrochloride (tacrine) in combination with dimethyl-(2,2,2-trichloro-1-hydroxyethyl)phosphonate (metrifonate).

19. The method according to claim 13, wherein said dementia is senile dementia.

20. The method of claim 13, wherein said dementia is senile dementia of the Alzheimer type.
